SEQUENCE LISTING (13 sequences)

SEQ ID NO: 1 - protein, 616 aa, Saccharomyces cerevisiae
MKNIISLVSKKKAASKNEDKNISESSRDIVNQQEVFNTENFEEGKKDSAFELDHLEFTTNSAQLGDSDEDNENVINETNTTDDANEANSEEKSMTLKQALLIYPKAALWSILVSTTLVMEGYDTALLNALYALPVFQRKFGTLNGEGSYEITSQWQIGLNMCVQCGEMIGLQITPYMVEFMGNRYTMITALGLLTAYVFILYYCKSLAMIAVGQVLSAMPWGCFQGLTVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQIDLTLKQIELTIEKERLLASKSGSFFDCFKGVNGRRTRLACLTWVAQNTSGACLLGYSTYFFERAGMATDKAFTFSVIQYCLGLAGTLCSWVISGRVGRWTILTYGLAFQMVCLFIIGGMGFGSGSGASNGAGGLLLALSFFYNAGIGAVVYCIVTEIPSAELRTKTIVLARICYNIMAVINAILTPYMLNVSDWNWGAKTGLYWGGFTAVTLAWVIIDLPETSGRTFSEINELFNQGVPARKFASTVVDPFGKGKTQHDSLADESISQSSSIKQRELNAADKC

SEQ ID NO: 2 - DNA, 1848 bp, Saccharomyces cerevisiae
atgaaaaatatcatttcattggtaagcaagaagaaggctgcctcaaaaaatgaggataaaaacatttctgagtcttcaagagatattgtaaaccaacaggaggttttcaatactgaaaattttgaagaagggaaaaaggatagtgcctttgagctagaccacttagagttcaccaccaattcagcccagttaggagattctgacgaagataacgagaatgtgattaatgagacgaacactactgatgatgcaaatgaagctaacagcgaggaaaaaagcatgactttaaagcaggcgttgctaatatatccaaaagcagccctgtggtccatattagtgtctactaccctggttatggaaggttatgataccgcactactgaacgcactgtatgccctgccagtttttcagagaaaattcggtactttgaacggggagggttcttacgaaattacttcccaatggcagattggtttaaacatgtgtgtccaatgtggtgagatgattggtttgcaaatcacgccttatatggttgaatttatggggaatcgttatacgatgattacagcacttggtttgttaactgcttatgtctttatcctctactactgtaaaagtttagctatgattgctgtgggacaagttctctcagctatgccatggggttgtttccagggtttgactgttacttatgcttcggaagtttgccctttagcattaagatattatatgaccagttactccaacatttgttggttatttggtcaaatcttcgcctctggtattatgaaaaactcacaagagaatttagggaactctgacttgggctataaattgccatttgctttacaatggatttggcctgctcctttaatgatcggtatctttttcgctcctgagtcgccctggtggttggtgagaaaggatagggtcgctgaggcaagaaaatctttaagcagaattttgagtggtaaaggcgccgagaaggacattcaaattgatcttactttaaagcagattgaattgactattgaaaaagaaagacttttagcatctaaatcaggatcattctttgattgtttcaagggagttaatggaagaagaacgagacttgcatgtttaacttgggtagctcaaaatactagcggtgcctgtttacttggttactcgacatatttttttgaaagagcaggtatggccaccgacaaggcgtttactttttctgtaattcagtactgtcttgggttagcgggtacactttgctcctgggtaatatctggccgtgttggtagatggacaatactgacctatggtcttgcatttcaaatggtctgcttatttattattggtggaatgggttttggttctggaagcggcgctagtaatggtgccggtggtttattgctggctttatcattcttttacaatgctggtatcggtgcagttgtttactgtatcgtaactgaaattccatcagcggagttgagaactaagactatagtgctggcccgtatttgctacaatatcatggccgttatcaacgctatattaacgccctatatgctaaacgtgagcgattggaactggggtgccaaaactggtctatactggggtggtttcacagcagtcactttagcttgggtcatcatcgatctgcctgagacaagtggtagaaccttcagtgaaattaatgaacttttcaaccaaggggttcctgccagaaaatttgcatctactgtggttgatccattcggaaagggaaaaactcaacatgattcgctagctgatgagagtatcagtcagtcctcaagcataaaacagcgagaattaaatgcagctgataaatgt

SEQ ID NO: 3 - protein, 593 aa, Saccharomyces cerevisiae
MKNIISLVSKKKAASKNEDKNISESSRDIVNQQEVFNTENFEEGKKDSAFELDHLEFTTNSAQLGDSDEDNENVINETNTTDDANEANSEEKSMTLKQALLIYPKAALWSILVSTTLVMEGYDTALLNALYALPVFQRKFGTLNGEGSYEITSQWQIGLNMCVQCGEMIGLQITPYMVEFMGNRYTMITALGLLTAYVFILYYCKSLAMIAVGQVLSAMPWGCFQGLTVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQIDLTLKQIELTIEKERLLASKSGSFFDCFKGVNGRRTRLACLTWVAQNTSGACLLGYSTYFFERAGMATDKAFTFSVIQYCLGLAGTLCSWVISGRVGRWTILTYGLAFQMVCLFIIGGMGFGSGSGASNGAGGLLLALSFFYNAGIGAVVYCIVTEIPSAELRTKTIVLARICYNIMAVINAILTPYMLNVSDWNWGAKTGLYWGGFTAVTLAWAIIDLPETTGRTFSEINELFNQGVPARKFASTVVDPFGKGKTQLIR

SEQ ID NO: 4 - DNA, 1848 bp, Saccharomyces cerevisiae
atgaaaaatatcatttcattggtaagcaagaagaaggctgcctcaaaaaatgaggataaaaacatttctgagtcttcaagagatattgtaaaccaacaggaggttttcaatactgaaaattttgaagaagggaaaaaggatagtgcctttgagctagaccacttagagttcaccaccaattcagcccagttaggagattctgacgaagataacgagaatgtgattaatgagacgaacactactgatgatgcaaatgaagctaacagcgaggaaaaaagcatgactttaaagcaggcgttgctaatatatccaaaagcagccctgtggtccatattagtgtctactaccctggttatggaaggttatgataccgcactactgaacgcactgtatgccctgccagtttttcagagaaaattcggtactttgaacggggagggttcttacgaaattacttcccaatggcagattggtttaaacatgtgtgtccaatgtggtgagatgattggtttgcaaatcacgccttatatggttgaatttatggggaatcgttatacgatgattacagcacttggtttgttaactgcttatgtctttatcctctactactgtaaaagtttagctatgattgctgtgggacaagttctctcagctatgccatggggttgtttccagggtttgactgttacttatgcttcggaagtttgccctttagcattaagatattatatgaccagttactccaacatttgttggttatttggtcaaatcttcgcctctggtattatgaaaaactcacaagagaatttagggaactctgacttgggctataaattgccatttgctttacaatggatttggcctgctcctttaatgatcggtatctttttcgctcctgagtcgccctggtggttggtgagaaaggatagggtcgctgaggcaagaaaatctttaagcagaattttgagtggtaaaggcgccgagaaggacattcaaattgatcttactttaaagcagattgaattgactattgaaaaagaaagacttttagcatctaaatcaggatcattctttgattgtttcaagggagttaatggaagaagaacgagacttgcatgtttaacttgggtagctcaaaatactagcggtgcctgtttacttggttactcgacatatttttttgaaagagcaggtatggccaccgacaaggcgtttactttttctgtaattcagtactgtcttgggttagcgggtacactttgctcctgggtaatatctggccgtgttggtagatggacaatactgacctatggtcttgcatttcaaatggtctgcttatttattattggtggaatgggttttggttctggaagcggcgctagtaatggtgccggtggtttattgctggctttatcattcttttacaatgctggtatcggtgcagttgtttactgtatcgtaactgaaattccatcagcggagttgagaactaagactatagtgctggcccgtatttgctacaatatcatggccgttatcaacgctatattaacgccctatatgctaaacgtgagcgattggaactggggtgccaaaactggtctatactggggtggtttcacagcagtcactttagcttgggccatcatcgatctgcctgagacaactggtagaaccttcagtgaaattaatgaacttttcaaccaaggggttcctgccagaaaatttgcatctactgtggttgatccattcggaaagggaaaaactcaactgattcgctagctgatgagagtatcagtcagtcctcaagcataaaacagcgagaattaaatgcagctgataaatgtt

SEQ ID NO: 5 - DNA, 729 bp, Saccharomyces cerevisiae
tgctgcataaagttaatgaattaagcaagtcaagagaagatggaacatcagaaccatagtacttctcctcgaaagagcactaattgtgctaaaaaaaaatatgaagtcttggacgttgtggcataagaagaatcgcgtttacctattatgagataattatggtcatattatgagataattatggtcatattatgctacgaatctgtgtctatattggtgaatttaccatgaaaaagtgatatttccggtacatgccattgaacggcttggcttaccttctcaattatcgtgcttggtttaaacgtttcttttgttccgcttctattttgttgtacttttcgcgcgaggaacaaggtttttttcctttgcctaaatatttgcctttgggttttggtcctccagagaatatcacgtactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaattttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatattataat

SEQ ID NO: 6 - DNA, 282 bp, Saccharomyces cerevisiae
taagtaaaagggttgtttttttttttttggaagaaataaggaatccctttgactgctcccaaaaccctcagctagctcgagattttatatttatacattttttatttttctgtaaaacatttatatttaccattttttaagcaaaatattgttagtagttagttaagatagcccaagcagcaatcaagcaaatatgagagtattttttctttagcacctggtacttgtgcctggatattgattcgaacaacatgccaggtcaaccgtattctcaattaactg

SEQ ID NO: 7 - protein, 394 aa, Saccharomyces cerevisiae
MKNIISLVSKKKAASKNEDKNISESSRDIVNQQEVFNTENFEEGKKDSAFELDHLEFTTNSAQLGDSDEDNENMINEMNATDEANEANSEEKSMTLKQALLKYPKAALWSILVSTTLVMEGYDTALLNALYALPVFQRKFGTLNGEGSYEITSQWQIGLNMCVQCGEMIGLQITTYMVEFMGNRYTMITALGLLTAYIFILYYCKSLAMIAVGQVLSAMPWGCFQGLTVTYASEVCPLALRYYMTSYSNICWLFGQIFASGIMKNSQENLGNSDLDYKLPFALQWIWPAPLMIGIFFAPESPWWLVRKDRVAEARKSLSRILSGKGAEKDIQVDLTLKQIELTIEKERLLASKSGSFFDCFKGVNGRRTRLACLAWVAQNTSGACLLGYSTYFF

SEQ ID NO: 8 - protein, 617 aa, Saccharomyces cerevisiae
MKNIISLVSKKKAASKNEDKNISESSRDIVNQQEVFNTEDFEEGKKDSAFELDHLEFTTNSAQLGDSDEDNENVINEMNATDDANEANSEEKSMTLKQALLKYPKAALWSILVSTTLVMEGYDTALLSALYALPVFQRKFGTLNGEGSYEITSQWQIGLNMCVLCGEMIGLQITTYMVEFMGNRYTMITALGLLTAYIFILYYCKSLAMIAVGQILSAIPWGCFQSLAVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQVDLTLKQIELTIEKERLLASKSGSFFNCFKGVNGRRTRLACLTWVAQNSSGAVLLGYSTYFFEKKQVMATDKAFTFSLIQYCLGLAGTLCSWVISGRVGRWTILTYGLAFQMVCLFIIGGMGFGSGSSASNGAGGLLLALSFFYNAGIGAVVYCIVAEIPSAELRTKTIVLARICYNLMAVINAILTPYMLNVSDWNWGAKTGLYWGGFTAVTLAWVIIDLPETTGRTFSEINELFNQGVPARKFASTVVDPFGKGKTQHDSLADESISQSSSIKQRELNAADKC

SEQ ID NO: 9 - protein, 616 aa, Saccharomyces cerevisiae
MKNIISLVSKKKAASKNEDKNISESSRDIVNQQEVFNTEDFEEGKKDSAFELDHLEFTTNSAQLGDSDEDNENVINEMNATDDANEANSEEKSMTLKQALLKYPKAALWSILVSTTLVMEGYDTALLSALYALPVFQRKFGTLNGEGSYEITSQWQIGLNMCVLCGEMIGLQITTYMVEFMGNRYTMITALGLLTAYIFILYYCKSLAMIAVGQILSAIPWGCFQSLAVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQVDLTLKQIELTIEKERLLASKSGSFFNCFKGVNGRRTRLACLTWVAQNSSGAVLLGYSTYFFERAGMATDKAFTFSLIQYCLGLAGTLCSWVISGRVGRWTILTYGLAFQMVCLFIIGGMGFGSGSSASNGAGGLLLALSFFYNAGIGAVVYCIVAEIPSAELRTKTIVLARICYNLMAVINAILTPYMLNVSDWNWGAKTGLYWGGFTAVTLAWVIIDLPETTGRTFSEINELFNQGVPARKFASTVVDPFGKGKTQHDSLADESISQSSSIKQRELNAADKC

SEQ ID NO: 10 - protein, 616 aa, Saccharomyces cerevisiae; identical to SEQ ID NO: 9

SEQ ID NO: 11 - protein, 616 aa, Saccharomyces cerevisiae; identical to SEQ ID NO: 9

SEQ ID NO: 12 - DNA, 40 bp, Saccharomyces cerevisiae
catatttttttgaaagagcaggtatggccaccgacaaggc

SEQ ID NO: 13 - DNA, 43 bp, Saccharomyces cerevisiae
catatttttttgaaaagaagcaggtaatggccaccgacaaggc

(19) United States
(12) Patent Application Publication
(10) Pub. No.: US 2014/0162335 A1
(43) Pub. Date: Jun. 12, 2014

(54) Recombinant Yeast Expressing AGT1

(71) Applicants: Pedro Esteban Bortiri, Research Triangle Park, NC (US); Richard Jason Hall, Research Triangle Park, NC (US)

(72) Inventors: Pedro Esteban Bortiri, Research Triangle Park, NC (US); Richard Jason Hall, Research Triangle Park, NC (US)

(21) Appl. No.: 14/127,566

(22) PCT Filed: Jun. 21, 2012
(86) PCT No.: PCT/US12/43518

§ 371 Date: Feb. 25, 2014

Related U.S. Application Data
(60) Provisional application No. 61/499,211, filed on Jun. 21, 2011.

(51) Int. Cl. C12P 7/06; C12N 15/81

(52) U.S. Cl. CPC C12P 7/06; C12N 15/81
USPC 435/161; 435/471; 435/254.2; 435/254.21; 435/254.23; 435/254.22

(57) ABSTRACT

The present invention relates to the identification of variants of the sugar transporter AGT1 that provide enhanced fermentation of oligosaccharides when recombinantly expressed in yeast. The invention further relates to polynucleotides encoding the variants, recombinant yeast cells expressing the variants, and use of the recombinant yeast cells to ferment oligosaccharides.

### FIELD OF THE INVENTION

The present invention relates to the identification of variants of the sugar transporter AGT1 (alpha-glucoside transporter-1) that provide enhanced fermentation of oligosaccharides when recombinantly expressed in yeast. The invention further relates to polynucleotides encoding the variants, recombinant yeast cells expressing the variants, and use of the recombinant yeast cells to ferment oligosaccharides.

### BACKGROUND OF THE INVENTION

With the ever increasing worldwide consumption of fossil fuels, there has been a corresponding interest in alternative energy options. Considerable interest has now been focused on the use of ethanol. Fuel ethanol could be made from crops which contain starch such as feed grains, food grains, and tubers, such as potatoes and sweet potatoes. Crops containing sugar, such as sugar beets, sugarcane, and sweet sorghum, also could be used for the production of ethanol. Sugar, in the form of raw or refined sugar, requires no pre-hydrolysis (unlike corn starch) prior to fermentation. Consequently, the process of producing ethanol from sugar is simpler than converting corn starch into ethanol. However, efficiently producing ethanol in sufficient quantities remains a concern.

Accordingly, it is desirable to design and develop new methods and systems for increasing the efficiency in the ethanol producing process. The present invention addresses previous shortcomings in the art by providing an improved fermentation process that enhances the level and rate of fermentation of oligosaccharides.

### SUMMARY OF THE INVENTION

The present invention is based, in part, on the identification of variants of AGT1 that enhance the level and/or rate of fermentation of oligosaccharides when the variants are recombinantly expressed in yeast. The invention is based further on the use of these variants to enhance the efficiency of fermentation of oligosaccharides by yeast.

Accordingly, as one aspect, the invention provides a method of fermenting an oligosaccharide to produce ethanol, comprising contacting the oligosaccharide with a recombinant yeast cell comprising a heterologous polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

In another aspect, the invention provides a method of modifying a yeast cell to decrease lag time for ethanol production during fermentation of an oligosaccharide, comprising inserting into the yeast cell a polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

In another aspect, the invention provides a method of modifying a yeast cell to increase the amount of ethanol production during fermentation of an oligosaccharide, comprising inserting into the yeast cell a polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

In a further aspect, the invention provides a recombinant yeast cell for production of ethanol from an oligosaccharide, the recombinant yeast cell comprising a heterologous polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

These and other aspects of the invention are set forth in more detail in the description of the invention below.

### BRIEF DESCRIPTION OF THE DRAWINGS

FIG. 1 shows Southern hybridization of yeast genomic DNA with a probe consisting of the amino acid-coding region of AGT1.

FIG. 2 shows the regions of MALI amplified and sequenced from eight yeast strains.

FIG. 3 shows a phylogenetic tree of AGT1 sequences.

FIG. 4 shows the fermentation of 4% isomaltulose (IM) by yeast strains in which the AGT1 gene has been fully sequenced.

FIG. 5 shows the fermentation of 4% IM by a ΔAGT1 yeast strain (lacking a native AGT1 gene) expressing variants of AGT1.

FIG. 6 shows the amount of ethanol produced by yeast carrying different AGT1-expressing cassettes as a function of hours of fermentation.

FIG. 7 shows the fermentation of 4% panose by strain 1334.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications, sequences identified by accession numbers, and other references cited herein are incorporated by reference in their entireties for the teachings relevant to the sentence and/or paragraph in which the reference is presented.

Unless the context indicates otherwise, it is specifically intended that the various features of the invention described herein can be used in any combination. For example, features described in relation to one embodiment may also be applicable to and combinable with other embodiments and aspects of the invention.

Moreover, the present invention also contemplates that in some embodiments of the invention, any feature or combination of features set forth herein can be excluded or omitted.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al., Current Protocols in Molecular Biology (Green Publishing Associates, Inc. and John Wiley & Sons, Inc., New York).

### I. DEFINITIONS

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, +10%, ±5%, ±1%, +0.5%, or even ±0.1% of the specified amount.

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in a biological activity of the polypeptide (e.g., sugar transporting activity or enhancement of fermentation) of at least about 50% or more as compared to the activity of a polypeptide consisting of the recited sequence.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a, cell in a pharmaceutically acceptable carrier. Thus, an isolated cell can be delivered to and/or introduced into a subject. In some embodiments, an isolated cell can be a cell that is removed from a subject and manipulated ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., at least 70%, 80%, 90%, 92%, 95%, 98%, or 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., at least 70%, 80%, 90%, 92%, 95%, 98%, or 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences. An “expression” vector refers to a viral or non-viral vector that is designed to express a product encoded by a heterologous nucleotide sequence inserted into the vector.

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

The term “heterologous” with respect to a polynucleotide means a polynucleotide that is not native to the cell in which it is located or, alternatively, a polynucleotide which is normally found in the cell but is in a different location than normal (e.g., in a vector or in a different location in the genome).

The term “recombinant yeast cell” refers to a yeast cell that comprises a heterologous polynucleotide. The heterologous polynucleotide may be inserted into the yeast cell by any means known in the art. In one embodiment, the polynucleotide is inserted by genetic engineering (e.g., insertion of an expression vector). In another embodiment, the polynucleotide is inserted by breeding (e.g., introgression).

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

As used herein, a “functional” polypeptide or “functional fragment” is one that substantially retains at least one biological activity normally associated with that polypeptide (e.g., sugar transport activity, enhancement of fermentation). In particular embodiments, the “functional” polypeptide or “functional fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as sugar transport activity and enhancement of fermentation can be measured using assays that are well known in the art and as described herein.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

The term “lag time,” as used herein, refers to the time from the first contact of oligosaccharide with the recombinant yeast cell to the time at which an increase in ethanol levels is first detected.

### II. RECOMBINANT YEAST EXPRESSING AGT1

AGT1 is a yeast protein that functions as a general α-glucoside transporter. The present invention is based in part on the discovery of AGT1 variants that are highly effective in enhancing the level and/or rate of fermentation of oligosaccharides to ethanol when the variants are recombinantly expressed in yeast.

Thus, one aspect of the invention provides a recombinant yeast cell for production of ethanol from an oligosaccharide, the recombinant yeast cell comprising a heterologous polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

Another aspect of the invention provides a method of modifying a yeast cell to decrease lag time for ethanol production during fermentation of an oligosaccharide, comprising inserting into the yeast cell a polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids. In some embodiments, the decreased lag time is in comparison to the lag time during fermentation with a yeast cell that does not express an AGT1 polypeptide of the invention.

In another aspect, the invention provides a method of modifying a yeast cell to increase the amount of ethanol production during fermentation of an oligosaccharide, comprising inserting into the yeast cell a polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids. In some embodiments, the increased amount of ethanol production is in comparison to the amount of ethanol production during fermentation with a yeast cell that does not express an AGT1 polypeptide of the invention.

(SEQ ID NO: 1)

| | |
|---|---|
| MKNIISLVSKKKAASKNEDKNISESSRDIVNQQEVFNTENFEEGK | 50 |
| KDSAF | |
| ELDHLEFTTNSAQLGDSDEDNENVINETNTTDDANEANSEEKSMT | 100 |
| LKQAL | |
| LIYPKAALWSILVSTTLVMEGYDTALLNALYALPVFQRKFGTLNG | 150 |
| EGSYE | |
| ITSQWQIGLNMCVQCGEMIGLQITPYMVEFMGNRYTMITALGLLT | 200 |
| AYVFI | |
| LYYCKSLAMIAVGQVLSAMPWGCFQGLTVTYASEVCPLALRYYMT | 250 |
| SYSNI | |
| CWLFGQIFASGIMKNSQENLGNSDLGYKLPFALQWIWPAPLMIGI | 300 |
| FFAPE | |
| SPWWLVRKDRVAEARKSLSRILSGKGAEKDIQIDLTLKQIELTIE | 350 |
| KERLL | |
| ASKSGSFFDCFKGVNGRRTRLACLTWVAQNTSGACLLGYSTYFFE | 400 |
| RAGMA | |
| TDKAFTFSVIQYCLGLAGTLCSWVISGRVGRWTILTYGLAFQMVC | 450 |
| LFIIG | |
| GMGFGSGSGASNGAGGLLLALSFFYNAGIGAVVYCIVTEIPSAEL | 500 |
| RTKTI | |
| VLARICYNIMAVINAILTPYMLNVSDWNWGAKTGLYWGGFTAVTL | 550 |
| AWVII | |
| DLPETSGRTFSEINELFNQGVPARKFASTVVDPFGKGKTQHDSLA | 600 |
| DESIS | |
| QSSSIKQRELNAADKC | 616 |

In some embodiments, the AGT1 polypeptide is at least 98%, 98.5%, 99%, 99.5%, or 100% identical to the amino acid sequence of SEQ ID NO:1. In one embodiment, the AGT1 polypeptide comprises, consists essentially of, or consists of the amino acid sequence of SEQ ID NO:1. In another embodiment, the AGT1 polypeptide comprises, consists essentially of, or consists of the amino acid sequence of SEQ ID NO:3.

(SEQ ID NO: 3)

| | |
|---|---|
| MKNIISLVSKKKAASKNEDKNISESSRDIVNQQEVFNTENFEEGK | 50 |
| KDSAF | |
| ELDHLEFTTNSAQLGDSDEDNENVINETNTTDDANEANSEEKSMT | 100 |
| LKQAL | |
| LIYPKAALWSILVSTTLVMEGYDTALLNALYALPVFQRKFGTLNG | 150 |
| EGSYE | |
| ITSQWQIGLNMCVQCGEMIGLQITPYMVEFMGNRYTMITALGLLT | 200 |
| AYVFI | |
| LYYCKSLAMIAVGQVLSAMPWGCFQGLTVTYASEVCPLALRYYMT | 250 |
| SYSNI | |
| CWLFGQIFASGIMKNSQENLGNSDLGYKLPFALQWIWPAPLMIGI | 300 |
| FFAPE | |
| SPWWLVRKDRVAEARKSLSRILSGKGAEKDIQIDLILKQIELTIE | 350 |
| KERLL | |
| ASKSGSFFDCFKGVNGRRTRLACLTWVAQNTSGACLLGYSTYFFE | 400 |
| RAGMA | |
| TDKAFTFSVIQYCLGLAGTLCSWVISGRVGRWTILTYGLAFQMVC | 450 |
| LFIIG | |
| GMGFGSGSGASNGAGGLLLALSFFYNAGIGAVVYCIVTEIPSAEL | 500 |
| RTKTI | |
| VLARICYNIMAVINAILTPYMLNVSDWNWGAKTGLYWGGFTAVTL | 550 |
| AWAII | |
| DLPETTGRTFSEINELFNQGVPARKFASTVVDPFGKGKTQLIR | 593 |

The AGT1 polypeptide includes functional portions or fragments (and polynucleotide sequences encoding the same) of at least about 590 amino acids starting from the N-terminus. In certain embodiments, the functional fragment can be about 590, 591, 592, 593, 594, 595, 596, 597, 598, 599, 600, 601, 602, 603, 604, 605, 606, 607, 608, 609, 610, 611, 612, 613, 614, 615, or 616 amino acids in length.

It has been discovered that an allele of AGT1 described in Han et al., Mol. Microbiol. 17:1093 (1995) (“the Han allele”) is ineffective in enhancing ethanol production during fermentation. The Han allele comprises the insertion of a single lysine residue after residue 396 of SEQ ID NO:1, as well as substitution of three additional amino acids at the following positions: lysine at position 396, glutamine at position 397, and valine at position 398 of SEQ ID NO:1. In certain embodiments, the AGT1 polypeptides of the invention exclude any sequence alterations (additions, subtractions and/or substitutions) at residues 390-405 of SEQ ID NO:1, e.g., residues 395-400. In one embodiment, the AGT1 polypeptides of the invention does not comprise an insertion of one or more amino acid residues at amino acid 396 of SEQ ID NO:1.

The present invention also encompasses AGT1 fusion polypeptides (and polynucleotide sequences encoding the same). For example, it may be useful to express the polypeptide (or functional fragment) as a fusion protein that can be recognized by a commercially available antibody (e.g., FLAG motifs) or as a fusion protein that can otherwise be more easily purified (e.g., by addition of a poly-His tail). Additionally, fusion proteins that enhance the stability of the polypeptide may be produced, e.g., fusion proteins comprising maltose binding protein (MBP) or glutathione-S-transferase. As another alternative, the fusion protein can comprise a reporter molecule. In other embodiments, the fusion protein can comprise a polypeptide that provides a function or activity that is the same as or different from the activity of the AGT1 polypeptide, e.g., a targeting, binding, or enzymatic activity or function.

Likewise, it will be understood that the polypeptides specifically disclosed herein will typically tolerate substitutions in the amino acid sequence and substantially retain biological activity. To identify polypeptides of the invention other than those specifically disclosed herein, amino acid substitutions may be based on any characteristic known in the art, including the relative similarity or differences of the amino acid side-chain substituents, for example, their hydrophobicity, hydrophilicity, charge, size, and the like.

Amino acid substitutions other than those disclosed herein may be achieved by changing the codons of the DNA sequence (or RNA sequence), according to the following codon table.

TABLE 1

| Amino Acid | | | Codons |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

In identifying amino acid sequences encoding polypeptides other than those specifically disclosed herein, the hydropathic index of amino acids may be considered. The importance of the hydropathic amino acid index in conferring interactive biologic function on a protein is generally understood in the art (see, Kyte and Doolittle, J. Mol. Biol. 157:105 (1982); incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, id.), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Accordingly, the hydropathic index of the amino acid (or amino acid sequence) may be considered when modifying the polypeptides specifically disclosed herein.

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Thus, the hydrophilicity of the amino acid (or amino acid sequence) may be considered when identifying additional polypeptides beyond those specifically disclosed herein.

In certain embodiments, the AGT1 polypeptide is encoded by a polynucleotide that is at least 80% identical to the nucleotide sequence of SEQ ID NO:2, e.g., at least 80%, 85%, 90%, 91%, 92%, 93%, 94%, 95%, 96%, 97%, 98%, 98.5%, 99%, 99.5%, or 100% identical to the nucleotide sequence of SEQ ID NO:2. In one embodiment, the polynucleotide comprises, consists essentially of, or consists of the nucleotide sequence of SEQ ID NO:2. In another embodiment, the polynucleotide comprises, consists essentially of, or consists of the nucleotide sequence of SEQ ID NO:4.

(SEQ ID NO: 2)
atgaaaaatatcatttcattggtaagcaagaagaaggctgcctcaaaaaatgaggataaaaacatttct
gagtcttcaagagatattgtaaaccaacaggaggttttcaatactgaaaattttgaagaagggaaaaag
gatagtgcctttgagctagaccacttagagttcaccaccaattcagcccagttaggagattctgacgaa
gataacgagaatgtgattaatgagacgaacactactgatgatgcaaatgaagctaacagcgaggaaaaa
agcatgactttaaagcaggcgttgctaatatatccaaaagcagccctgtggtccatattagtgtctact
accctggttatggaaggttatgataccgcactactgaacgcactgtatgccctgccagtttttcagaga
aaattcggtactttgaacggggagggttcttacgaaattacttcccaatggcagattggtttaaacatg
tgtgtccaatgtggtgagatgattggtttgcaaatcacgccttatatggttgaatttatggggaatcgt
tatacgatgattacagcacttggtttgttaactgcttatgtctttatcctctactactgtaaaagttta
gctatgattgctgtgggacaagttctctcagctatgccatggggttgtttccagggtttgactgttact
tatgcttcggaagtttgccctttagcattaagatattatatgaccagttactccaacatttgttggtta
tttggtcaaatcttcgcctctggtattatgaaaaactcacaagagaatttagggaactctgacttgggc
tataaattgccatttgctttacaatggatttggcctgctcctttaatgatcggtatctttttcgctcct
gagtcgccctggtggttggtgagaaaggatagggtcgctgaggcaagaaaatctttaagcagaattttg
agtggtaaaggcgccgagaaggacattcaaattgatcttactttaaagcagattgaattgactattgaa
aaagaaagacttttagcatctaaatcaggatcattctttgattgtttcaagggagttaatggaagaaga
acgagacttgcatgtttaacttgggtagctcaaaatactagcggtgcctgtttacttggttactcgaca
tatttttttgaaagagcaggtatggccaccgacaaggcgtttactttttctgtaattcagtactgtctt
gggttagcgggtacactttgctcctgggtaatatctggccgtgttggtagatggacaatactgacctat
ggtcttgcatttcaaatggtctgcttatttattattggtggaatgggttttggttctggaagcggcgct
agtaatggtgccggtggtttattgctggctttatcattcttttacaatgctggtatcggtgcagttgtt
tactgtatcgtaactgaaattccatcagcggagttgagaactaagactatagtgctggcccgtatttgc
tacaatatcatggccgttatcaacgctatattaacgccctatatgctaaacgtgagcgattggaactgg
ggtgccaaaactggtctatactggggtggtttcacagcagtcactttagcttgggtcatcatcgatctg
cctgagacaagtggtagaaccttcagtgaaattaatgaacttttcaaccaaggggttcctgccagaaaa
tttgcatctactgtggttgatccattcggaaagggaaaaactcaacatgattcgctagctgatgagagt
atcagtcagtcctcaagcataaaacagcgagaattaaatgcagctgataaatgt
(SEQ ID NO: 4)
atgaaaaatatcatttcattggtaagcaagaagaaggctgcctcaaaaaatgaggataaaaacatttct
gagtcttcaagagatattgtaaaccaacaggaggttttcaatactgaaaattttgaagaagggaaaaag
gatagtgcctttgagctagaccacttagagttcaccaccaattcagcccagttaggagattctgacgaa
gataacgagaatgtgattaatgagacgaacactactgatgatgcaaatgaagctaacagcgaggaaaaa
agcatgactttaaagcaggcgttgctaatatatccaaaagcagccctgtggtccatattagtgtctact
accctggttatggaaggttatgataccgcactactgaacgcactgtatgccctgccagtttttcagaga
aaattcggtactttgaacggggagggttcttacgaaattacttcccaatggcagattggtttaaacatg
tgtgtccaatgtggtgagatgattggtttgcaaatcacgccttatatggttgaatttatggggaatcgt
tatacgatgattacagcacttggtttgttaactgcttatgtctttatcctctactactgtaaaagttta
gctatgattgctgtgggacaagttctctcagctatgccatggggttgtttccagggtttgactgttact
tatgcttcggaagtttgccctttagcattaagatattatatgaccagttactccaacatttgttggtta
tttggtcaaatcttcgcctctggtattatgaaaaactcacaagagaatttagggaactctgacttgggc
tataaattgccatttgctttacaatggatttggcctgctcctttaatgatcggtatctttttcgctcct
gagtcgccctggtggttggtgagaaaggatagggtcgctgaggcaagaaaatctttaagcagaattttg
agtggtaaaggcgccgagaaggacattcaaattgatcttactttaaagcagattgaattgactattgaa
aaagaaagacttttagcatctaaatcaggatcattctttgattgtttcaagggagttaatggaagaaga
acgagacttgcatgtttaacttgggtagctcaaaatactagcggtgcctgtttacttggttactcgaca
tatttttttgaaagagcaggtatggccaccgacaaggcgtttactttttctgtaattcagtactgtctt
gggttagcgggtacactttgctcctgggtaatatctggccgtgttggtagatggacaatactgacctat
ggtcttgcatttcaaatggtctgcttatttattattggtggaatgggttttggttctggaagcggcgct
agtaatggtgccggtggtttattgctggctttatcattcttttacaatgctggtatcggtgcagttgtt
tactgtatcgtaactgaaattccatcagcggagttgagaactaagactatagtgctggcccgtatttgc
tacaatatcatggccgttatcaacgctatattaacgccctatatgctaaacgtgagcgattggaactgg
ggtgccaaaactggtctatactggggtggtttcacagcagtcactttagcttgggccatcatcgatctg
cctgagacaactggtagaaccttcagtgaaattaatgaacttttcaaccaaggggttcctgccagaaaa
tttgcatctactgtggttgatccattcggaaagggaaaaactcaactgattcgctagctgatgagagta
tcagtcagtcctcaagcataaaacagcgagaattaaatgcagctgataaatgtt

In embodiments of the invention, the polynucleotide encoding the AGT1 polypeptide (or functional fragment) will hybridize to the nucleic acid sequences specifically disclosed herein or fragments thereof under standard conditions as known by those skilled in the art and encode a functional polypeptide or functional fragment thereof.

For example, hybridization of such sequences may be carried out under conditions of reduced stringency, medium stringency or even stringent conditions (e.g., conditions represented by a wash stringency of 35-40% formamide with 5×Denhardt's solution, 0.5% SDS and 1×SSPE at 37° C.; conditions represented by a wash stringency of 40-45% formamide with 5×Denhardt's solution, 0.5% SDS, and 1×SSPE at 42° C.; and conditions represented by a wash stringency of 50% formamide with 5×Denhardt's solution, 0.5% SDS and 1×SSPE at 42° C., respectively) to the polynucleotide sequences encoding the AGT1 polypeptide or functional fragments thereof specifically disclosed herein. See, e.g., Sambrook et al., Molecular Cloning: A Laboratory Manual 2nd Ed. (Cold Spring Harbor, N.Y., 1989).

Further, it will be appreciated by those skilled in the art that there can be variability in the polynucleotides that encode the AGT1 polypeptides (and fragments thereof) of the present invention due to the degeneracy of the genetic code. The degeneracy of the genetic code, which allows different nucleic acid sequences to code for the same polypeptide, is well known in the literature (See, e.g., Table 1).

As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity with respect to the coding sequence of the polypeptides disclosed herein is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

The polynucleotide encoding the AGT1 polypeptide of the invention may be inserted into a yeast cell as part of an episomal vector and/or integrated into the genome. Multiple copies of the polynucleotide can be inserted into the cell, e.g., up to 10 copies or more, e.g., up to 100 copies or more.

In one embodiment, the polynucleotide is in an expression vector that is maintained episomally and thus comprises a sequence for autonomous replication. The expression vector may be one that maintains a single copy per cell (e.g., a vector comprising a CEN/ARS origin of replication) or one that maintains multiple copies per cell (e.g., a vector comprising a 2μ origin of replication). For example, the following vectors may be selected: (a) a replicative vector (YEp) at high copy number having a replication origin in yeast (e.g., YEplac181); (b) a replicative vector (YRp) at high copy number having a chromosomal ARS sequence as a replication origin; (c) a linear replicative vector (YLp) at high copy number having a telomer sequence as a replication origin; and (d) a replicative vector (YCp) at low copy number having a chromosomal ARS and centromere sequences.

In another embodiment, the polynucleotide is integrated in one or more copies into the genome of the host cell. Integration into the host cell's genome may be by homologous recombination as is well known in the art of fungal molecular genetics (see, e.g., WO 90/14423, EP-A-0 481 008, EP-A-0 635 574 and U.S. Pat. No. 6,265,186). For example, an integrative vector (YIp) possessing no origin in the host cells may be selected for use in homologous recombination.

The polynucleotides encoding the polypeptides of the invention will typically be associated with the necessary regulatory sequences for the transcription and translation of the inserted protein sequence(s). In particular, the expression vector may include promoter and terminator sequences for promoting and terminating transcription of the gene in the transformed yeast cell and expressing the AGT1 polypeptide. Examples of regulatory sequences which may be used in a nucleic acid molecule of the invention include the promoters and terminators of genes for alcohol dehydrogenase I (ADHI), glyceraldehyde-3-phosphate dehydrogenase (GAPDH), 3-phosphoglycerate kinase (PGK), triose phosphate isomerase (TPI), phosphofructokinase (PPK), pyruvate kinase (PYK), GAL1, GAL4, GAL10, CUP1, GAP, CYC1, PHO5, HIS3, ADC1, TRP1, URA3, LEU2, ENO, AOX1, or other promoters that are functional in yeast. In certain embodiments, the promoter is one that is insensitive to catabolite (glucose) repression. In other embodiments, the promoter and terminator may be the ones associated with an endogenous AGT1 gene. Examples include the promoter and terminator from the AGT1 gene in yeast strain 1334.

Promoter of AGT1 from 1334
(SEQ ID NO: 5)
tgctgcataaagttaatgaattaagcaagtcaagagaagatggaacatcagaaccatagtacttctcct
cgaaagagcactaattgtgctaaaaaaaaatatgaagtcttggacgttgtggcataagaagaatcgcgt
ttacctattatgagataattatggtcatattatgagataattatggtcatattatgctacgaatctgtg
tctatattggtgaatttaccatgaaaaagtgatatttccggtacatgccattgaacggcttggcttacc
ttctcaattatcgtgcttggtttaaacgtttcttttgttccgcttctattttgttgtacttttcgcgcg
aggaacaaggtttttttcctttgcctaaatatttgcctttgggttttggtcctccagagaatatcacgt
actatggcagcgaaaggagctttaaggttttaattaccocatagccatagattctactcggtctatcta
tcatgtaacactccgttgatgcgtactagaaaatgacaacgtaccgggcttgagggacatacagagaca
attacagtaatcaagagtgtacccaattttaacgaactcagtaaaaaataaggaatgtcgacatcttaa
ttttttatataaagcggtttggtattgattgtttgaagaattttcgggttggtgtttctttctgatgct
acatagaagaacatcaaacaactaaaaaaatattataat
Terminator of AGT1 from 1334
(SEQ ID NO: 6)
Taagtaaaagggttgtttttttttttttggaagaaataaggaatccctttgactgctcccaaaaccctc
agctagctcgagattttatatttatacattttttatttttctgtaaaacatttatatttaccatttttt
aagcaaaatattgttagtagttagttaagatagcccaagcagcaatcaagcaaatatgagagtattttt
tctttagcacctggtacttgtgcctggatattgattcgaacaacatgccaggtcaaccgtattctcaat
taactg

Optionally, a selectable marker may be present in the vector. As used herein, the term “marker” refers to a gene or nucleotide sequence encoding a trait or a phenotype which permits the selection of, or the screening for, a host cell containing the marker. The marker gene or nucleotide sequence may be an antibiotic resistance gene or nucleotide sequence whereby the appropriate antibiotic can be used to select for transformed cells from among cells that are not transformed. Examples of suitable antibiotic resistance markers include, e.g., dihydrofolate reductase, hygromycin-B-phosphotransferase, 3′-O-phosphotransferase II (kanamycin, neomycin and G418 resistance). Alternatively, non-antibiotic resistance markers can be used, such as auxotrophic markers (URA3, TRP1, LEU2) or the S. pombe TPI gene (described by Russell, Gene 40:125 (1985)). In certain embodiments the host cells transformed with the vectors are marker gene free. Methods for constructing recombinant marker gene free microbial host cells are disclosed in EP 0635574 and are based on the use of bidirectional markers such as the A. nidulans amdS (acetamidase) gene or the yeast URA3 and LYS2 genes. Alternatively, a screenable marker such as Green Fluorescent Protein, lacZ, luciferase, chloramphenicol acetyltransferase, and/or beta-glucuronidase may be incorporated into the vectors of the invention, allowing for screening of transformed cells.

Optional further elements that may be present in the vectors of the invention include, but are not limited to, one or more leader sequences, enhancers, integration factors, and/or reporter genes, intron sequences, centromers, telomers and/or matrix attachment (MAR) sequences.

The transformation of yeast cells with vectors can be carried out according to the methods generally used in genetic engineering and biological engineering such as the spheroplast method (e.g., Proc. Natl. Acad. Sci. USA, 75:1929 (1978)), the lithium acetate method (e.g., J. Bacteriol, 153:163 (1983)), and the electroporation method (e.g., Methods in Enzymology, 194:182 (1991)).

An alternative to the recombinant approach of transforming yeast cells with an AGT1-carrying expression plasmid or integrating the expression cassette in a yeast chromosomal location consists of introgressing or breeding a select AGT1 gene into a desired genetic background such as those possessed by elite industrial strains. Crossing S. cerevisiae and other yeast is a widely practiced technique, described in general in many books. As an example, the following steps can be used to introgress the AGT1 gene from one yeast strain, named A, into another strain that either lacks AGT1 or has an AGT1 allele with undesired characteristics, named strain B:
1. Transform each strain with plasmids carrying selection to different drugs, for example transform strain A with a plasmid carrying kanMX4 for selection on G418 and strain B with a plasmid carrying the marker hphMX4 for selection against hygromycin;
2. Sporulate both strains;
3. Mate transformed strains A and B and select on medium containing both drugs, in this case G418 and hygromycin;
4. Sporulate and genotype spores to select for those that carry the desired AGT1 allele; and
5. Repeat the crossing strategy to keep introgressing the AGT1 allele into the desired background.

The yeast cell may be from any strain of yeast that is known to or has the potential to ferment oligosaccharides into ethanol. In one embodiment, the yeast is selected from the group consisting of Saccharomyces, Schizosaccharomyces, Kluyveromyces, Trichosporon, Schwanniomyces, Pichia, Hansenula, Arxula, Candida, Kloeckera, and Yarrowia. In another embodiment, the yeast is Saccharomyces cerevisiae. The yeast can be one that does not comprise a functional endogenous AGT1 gene.

In one embodiment, the yeast cell is one that naturally does not contain an AGT1 gene. In another embodiment, the yeast cell is one in which the endogenous AGT1 gene has been inactivated, e.g., due to a partial or complete deletion of the endogenous gene or replacement of some or all of the endogenous gene with a polynucleotide encoding the AGT1 polypeptide of the invention. The term inactivation of the gene as used herein refers to the lowering or loss of functions inherent in the gene or the polypeptide encoded by the gene induced by various techniques for genetic engineering or biological engineering; for example, gene disruption (e.g., Methods in Enzymology 194:281 (1991)), introduction of a movable genetic element into the gene (e.g., Methods in Enzymology 194:342 (1991)), introduction and expression of the antisense gene (e.g., Japanese Published Examined Patent Application No. 40943/95, and The 23rd European Brewery Cony. Proc., 297-304 (1991)), and introduction of DNA relating to silencing to the vicinity of the gene (e.g., Cell 75:531 (1993)).

### III. FERMENTATION OF OLIGOSACCHARIDES

The recombinant yeast cell of the invention can be use to ferment oligosaccharides at enhanced levels and/or rates. Thus, one aspect of the invention provides a method of fermenting an oligosaccharide to produce ethanol, comprising contacting the oligosaccharide with a recombinant yeast cell comprising a heterologous polynucleotide encoding a yeast AGT1 polypeptide; wherein the yeast AGT1 polypeptide comprises an amino acid sequence that is at least 98% identical to the amino acid sequence of SEQ ID NO:1 or an N-terminal fragment thereof of at least about 590 amino acids.

The oligosaccharide can be any oligosaccharide that can be transported into the cell by AGT1. In certain embodiments, the oligosaccharide is one with an α-glucoside linkage. In one embodiment, the oligosaccharide is a disaccharide or trisaccharide. In another embodiment, the oligosaccharide is selected from the group consisting of isomaltulose, trehalulose, maltose, panose, and maltotriose. In a further embodiment, the oligosaccharide is isomaltulose or trehalulose. In another embodiment, the oligosaccharide is panose. In certain embodiments, the oligosaccharide is not maltose. In other embodiments, the oligosaccharide is not maltotriose. In further embodiments, the oligosaccharide is neither maltose nor maltotriose.

The oligosaccharide to be fermented can be from any source. In certain embodiments, the oligosaccharide is obtained from plant material. In one embodiment, the oligosaccharide is from a plant that accumulates large amounts of sugar, e.g., sugar beet, sorghum, or sugarcane. In another embodiment, the oligosaccharide is from the cellulosic material of a plant (e.g., maize) that has been hydrolyzed to oligosaccharides. In certain embodiments, the oligosaccharide is from a plant that has been modified to accumulate higher levels of oligosaccharides, e.g., isomaltulose and/or trehalulose, such as is described in WO 2009/152285, herein incorporated by reference in its entirety.

In certain embodiments, the fermentation occurs at a rate that is faster than the rate when a yeast cell that does not contain the AGT1 polypeptide of the invention is used. The rate of fermentation may be 10%, 20%, 30%, 40%, 50%, 60%, 70%, 80%, 90%, 100%, 125%, 150%, or 200% or more faster than the rate when a yeast cell that does not contain the AGT1 polypeptide of the invention is used. In other embodiments, the production of ethanol during fermentation occurs with a shorter lag time than occurs when a yeast cell that does not contain the AGT1 polypeptide of the invention is used. The lag time may be 10%, 20%, 30%, 40%, 50%, 60%, 70%, 80%, 90%, 100%, 125%, 150%, or 200% or more shorter than the rate when a yeast cell that does not contain the AGT1 polypeptide of the invention is used. In one embodiment, the amount of ethanol produced during fermentation reaches half maximum within 15 hours (e.g., within 10 hours) of contacting the oligosaccharide with the recombinant yeast cell. In other embodiments, the amount of ethanol produced during fermentation is higher than the amount produced using a yeast cell that does not contain the AGT1 polypeptide of the invention. The amount of ethanol produced may be 10%, 20%, 30%, 40%, 50%, 60%, 70%, 80%, 90%, 100%, 125%, 150%, or 200% or more higher than the amount produced when a yeast cell that does not contain the AGT1 polypeptide of the invention is used.

The fermentation can be carried out by any process known in the art and described herein. The fermentation process may be an aerobic or an anaerobic fermentation process. An anaerobic fermentation process is herein defined as a fermentation process run in the absence of oxygen or in which substantially no oxygen is consumed, e.g., less than 5 mmol/L/h, and wherein organic molecules serve as both electron donor and electron acceptors.

The fermentation process is preferably run at a temperature that is optimal for the recombinant yeast. Thus, for most yeasts, the fermentation process is performed at a temperature which is less than 38° C. For yeast cells, the fermentation process is preferably performed at a temperature which is lower than 35, 33, 30 or 28° C. and at a temperature which is higher than 20, 22, or 25° C.

The present invention is more particularly described in the following examples that are intended as illustrative only since numerous modifications and variations therein will be apparent to those skilled in the art.

### Example 1

### Materials and Methods

Standard recombinant DNA and molecular cloning techniques used herein are well known in the art and are described by J. Sambrook, E. F. Fritsch and T. Maniatis, Molecular Cloning: A Laboratory Manual, Cold Spring Harbor Laboratory, Cold Spring Harbor, N.Y. (1989); T. J. Silhavy, M. L. Berman, and L. W. Enquist, Experiments with Gene Fusions, Cold Spring Harbor Laboratory, Cold Spring Harbor, N.Y. (1984); and by Ausubel, F. M. et al., Current Protocols in Molecular Biology, pub. by Greene Publishing Assoc. and Wiley-Interscience (1987). Yeast growth and manipulations were done following published protocols (D. C. Amberg, D. J. Burke, J. N. Strathern, Methods in Yeast Genetics: A Cold Springs Harbor Laboratory Course Manual. D. C. Amberg, D. J. Burke, J. N. Strathern, Ed., (Cold Springs Harbor Laboratory Press, Cold Springs Harbor, 2005); I. Stansfield, M. J. R. Stark, in Methods in Microbiology, I. S. M. J. R. Stansfield, Ed. (ELSEVIER ACADEMIC PRESS INC, 525 B Street, Suite 1900, San Diego, Calif. 92101-4495 USA, 2007), vol. 36).

All strains are Saccharomyces cerevisiae and were obtained from ATCC (204802 and BJ5464) and DSMZ (1884 and 1334). The strain carrying a deletion of AGT1 (ΔAGT1) was obtained from the haploid ORF deletion library (GSA-4, ATCC). Plasmids pGEM30, p416 MET25, and p426 MET25 were obtained from ATCC. The kanMX4 cassette was amplified by polymerase chain reaction (PCR) from a yeast strain carrying a deletion of the HO locus (GSA-7, ATCC).

The AGT1 fragments were obtained by PCR amplification from strains 1334 (AGT11334 and natAGT11334) and 204802 (AGT1802). The natAGT1334 expression cassette included the promoter, CDS, and transcriptional terminator of AGT1 from strain 1334. The AGT1Han allele was synthesized by GeneArt from GenBank Accession Number L47346 (Han et al., Mol. Microbiol. 17:1093 (1995)). AGT11334, AGT1802, and AGT1Han consisted of the CDS of AGT1 cloned between the promoter and terminator of the triose phosphate isomerase gene (TPI).

Each AGT1 expression cassette (promoter-CDS-terminator) was cloned into three plasmids. The first two have a ura3 gene as a selectable marker and were derived from the plasmids p416 MET25 and p426 MET25 by replacement of the expression cassette. p416 MET25 has a CEN/ARS yeast origin of replication, which maintains a single copy of the plasmid per cell. p426 MET25 has a 2u origin of replication, for multiple copy number of the plasmid per cell. The third plasmid has a CEN/ARS origin of replication and a kanMX4 selection marker and was derived from pGEM30.

Transformations of 204802, ΔAGT1, and BJ5464 were done using the FAST™-Yeast Transformation kit (G-Biosciences, St. Louis, Mo., USA), following the manufacturer's instructions. Transformation of strain 1848 was done using electroporation (Thompson et al., Yeast 14:565 (1998)).

Following transformation, yeast cells were plated on medium containing appropriate selection (synthetic medium without uracil for ura3 constructs or YPD plus G418 (Sigma) for kanMX4 plasmids) and colonies were screened by PCR to confirm the presence of the expression cassette. Two or three clones were grown overnight in 5 ml of either synthetic medium without uracil or YPD with 200 μg/ml of G418. Both media were supplemented with 4% isomaltulose. This overnight culture was used to inoculate 45 ml of the same medium for the fermentation test. Production of ethanol was monitored every 10 minutes as a function of volume loss due to CO₂ production by a weighting robot over the course of 50 hours. Tables 3 and 4 below are the estimates for ethanol produced at the end of the 50 hours.

### Example 2

### Natural Diversity of AGT1 in Yeast

In order to identify alleles of AGT1 that may confer superior IM fermentation, this gene was characterized from a number of yeast strains by sequencing and nucleic acid blotting (Southern). AGT1 is a single copy gene present in most yeast strains.

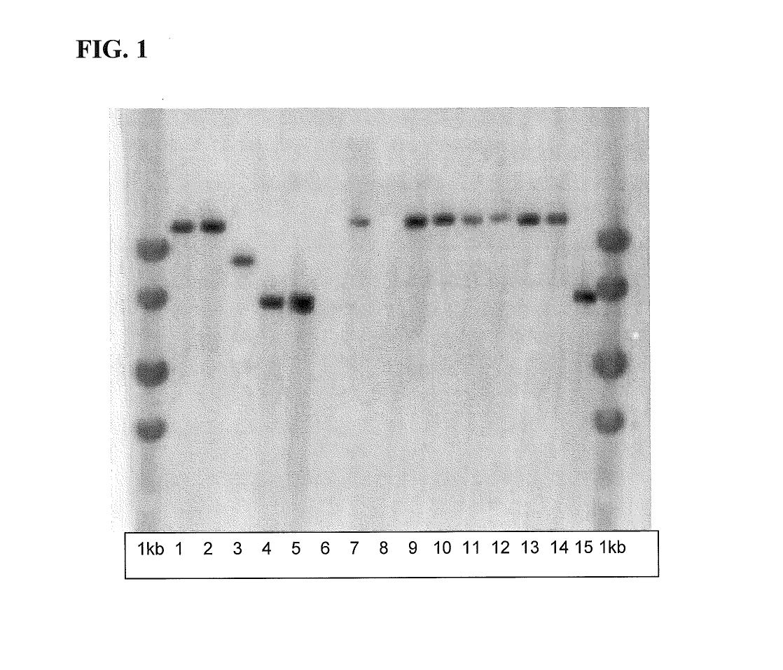

A Southern hybridization of DNA from 15 strains of yeast shows that all but two strains carry a copy of AGT1 (FIG. 1). Strains are 1: 3798; 2: 3799; 3: 1848; 4: 1334; 5: 9763; 6: Ethanol Red; 7: 204802; 8: 201149; 9: 42335; 10: 495; 11: 204802; 12: 475; 13: 200060; 14: 208023; and 15: commercial baking yeast. Genomic DNA lanes are flanked by 1 kb marker. One of the strains lacking AGT1 is Ethanol Red, a null fermenter of IM.

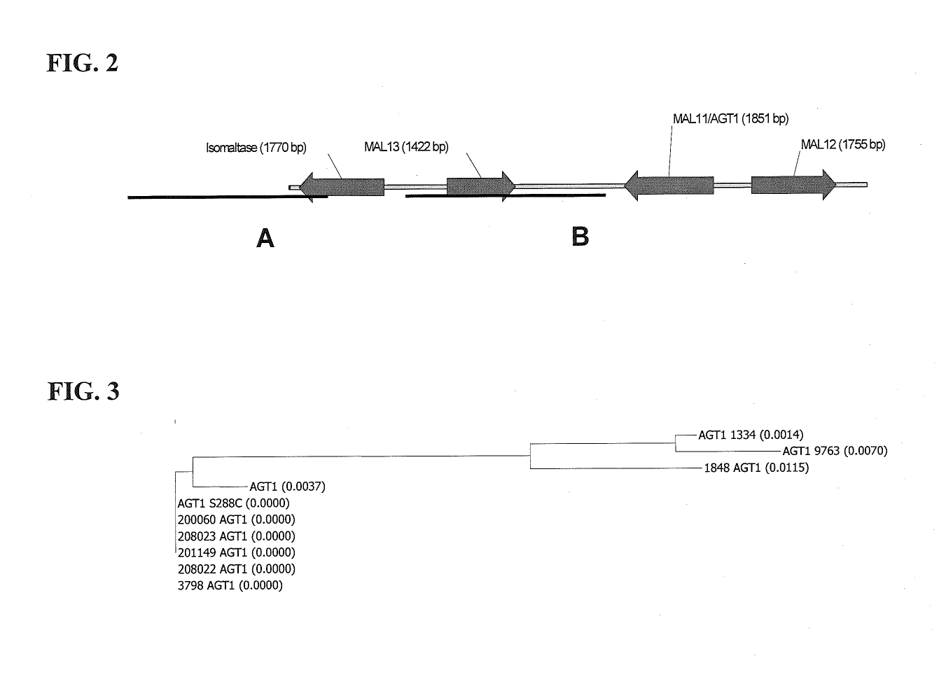

A number of yeast strains that are poor or null fermenters of IM carry a copy of AGT1, like strain 1848. To see if AGT1 sequences might explain IM fermentation phenotypes, a number of yeast strains were selected with different IM fermentation performance, and two regions were sequenced, A and B (FIG. 2), encompassing the genes IMA1, MAL13, MAL12 and AGT1. AGT1 sequences from strains 1334 and 9763 were initially obtained by amplification of just the open reading frame (coding sequence).

Because region B could not be amplified from strains 1334 and 9763, their genomes were sequenced and a contig comprising the AGT1 ORF and 761 by of upstream and 282 by of downstream regulatory sequence was obtained for 1334. The assembled contig was confirmed by performing PCR amplification, cloning and sequencing of strain 1334.

The amino acid sequence of AGT1 from several yeast strains is shown below. A phylogenetic tree of the AGT1 sequences is shown in FIG. 3.

Yeast AGT1 Sequences

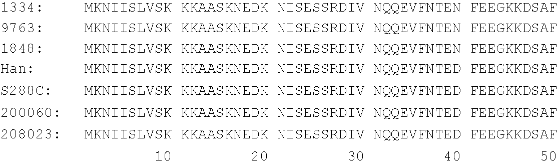

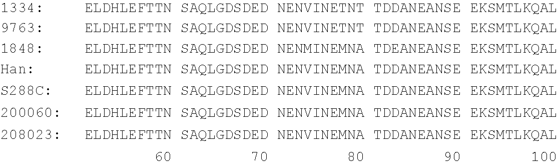

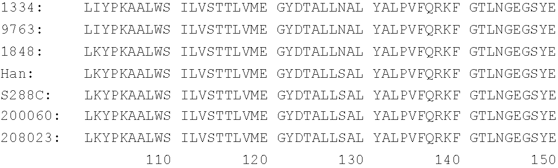

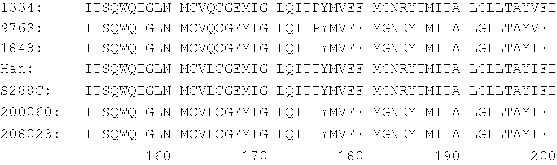

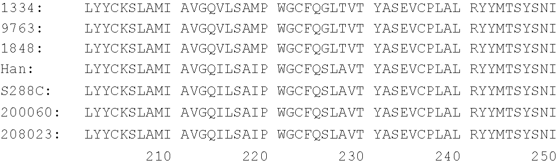

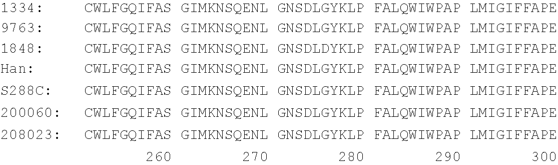

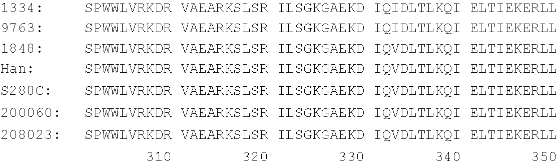

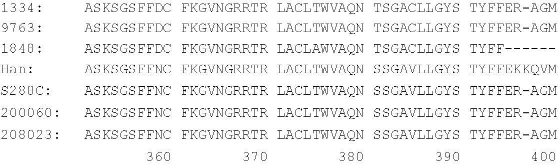

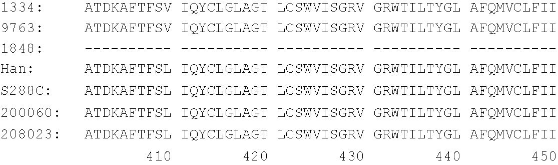

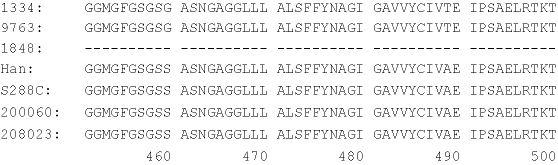

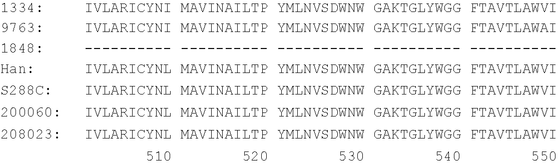

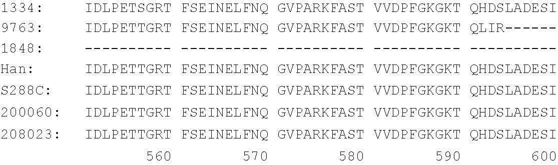

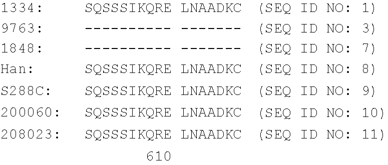

Most strains have an AGT1 protein consisting of 616 amino acids. The AGT1Han allele is 617 amino acids long (Han et al., Mol. Microbiol. 17:1093 (1995)) and there are two strains that have early stop codons, 9763 and 1848. AGT1 from strain 9763 (AGT19763) is very similar to AGT11334 but its sequence is 26 amino acids shorter. The amino acid sequence of AGT19763 is greater than 99% identical to AGT11334. In contrast, the AGT1 sequences from S288C, 200060, and 208023 are only 97% identical to AGT11334. The AGT1Han amino acid sequence, in addition to being less than 97% identical to AGT1134, also contains a single amino acid insertion after residue 396.

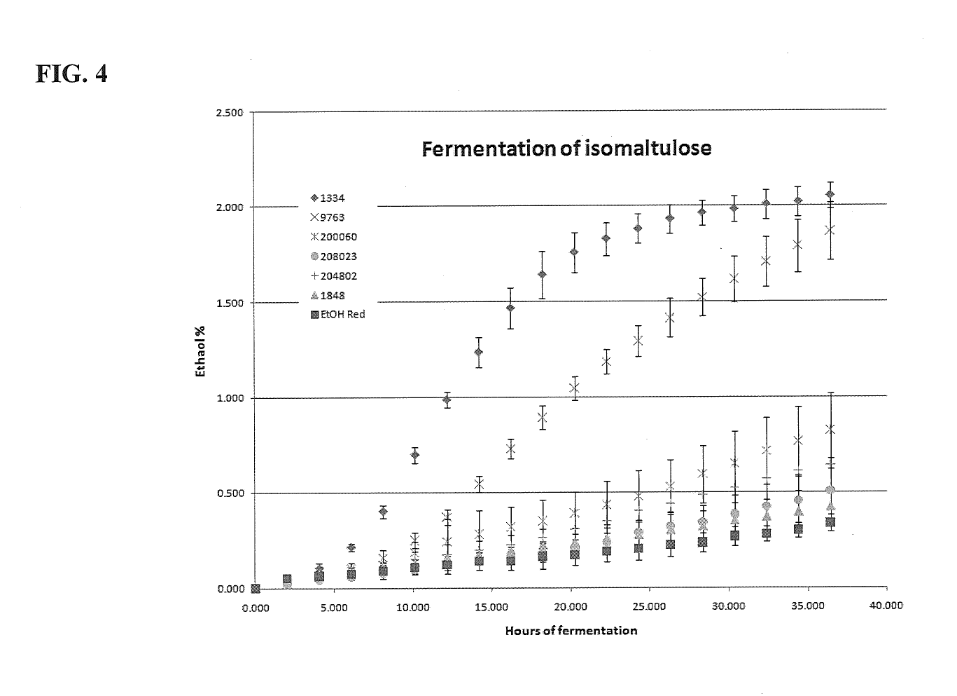

The fermentation performance of the strains for which AGT1 was fully sequenced was tested. FIG. 4 and Table 2 show the production of ethanol from 4% IM. The averages and standard deviations are from triplicates. Strains 1334 and 9763 are good fermenters of TM but 1334 is considerably better. AGT11848 is much shorter, only 394 amino acids, and this strain is almost a null fermenter. From this data it is likely that the group that includes AGT19763, AGT11334, and perhaps AGT11848 contains substitutions that confer superior IM fermentation and the differences in fermentation, especially AGT19763 vs. AGT11334, are due to early terminations in the protein sequence.

### Example 3

### Expression of natAGT1₁₃₃₄ in Three Yeast Strains Increases Isomaltulose Fermentation

Three strains (1884, 204802, and BJ5464) were transformed with a plasmid carrying the expression cassette natAGT11334 and a CEN/ARS origin of replication. Selection was done by growing the transformed yeast in medium containing 200 μg/ml of G418. Results are shown in Table 3, where EV corresponds to empty vector control, AGT1 is yeast expressing natAGT11334, and 1848, 204802, and BJ5464 are three yeast strains.

TABLE 2

| Ferm | 1334 | | EtOH Red | | 1848 | | 9763 | | 200060 | | 208023 | | 204802 | |
|---|---|---|---|---|---|---|---|---|---|---|---|---|---|---|
| Hours | Ave | Stdev | Ave | Stdev | Ave | Stdev | Ave | Stdev | Ave | Stdev | Ave | Stdev | Ave | Stdev |
| 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 2.023 | 0.045 | 0.011 | 0.051 | 0.022 | 0.038 | 0.000 | 0.025 | 0.011 | 0.032 | 0.011 | 0.026 | 0.011 | 0.032 | 0.022 |
| 4.047 | 0.103 | 0.029 | 0.064 | 0.029 | 0.057 | 0.000 | 0.064 | 0.029 | 0.064 | 0.011 | 0.045 | 0.011 | 0.070 | 0.029 |
| 6.070 | 0.212 | 0.019 | 0.076 | 0.033 | 0.070 | 0.011 | 0.102 | 0.029 | 0.089 | 0.011 | 0.057 | 0.000 | 0.108 | 0.058 |
| 8.093 | 0.399 | 0.031 | 0.089 | 0.040 | 0.108 | 0.029 | 0.159 | 0.040 | 0.121 | 0.011 | 0.077 | 0.000 | 0.115 | 0.069 |
| 10.116 | 0.695 | 0.040 | 0.108 | 0.040 | 0.133 | 0.057 | 0.248 | 0.038 | 0.185 | 0.055 | 0.096 | 0.000 | 0.153 | 0.069 |
| 12.140 | 0.985 | 0.042 | 0.121 | 0.048 | 0.159 | 0.067 | 0.370 | 0.040 | 0.242 | 0.122 | 0.121 | 0.011 | 0.172 | 0.069 |
| 14.163 | 1.236 | 0.079 | 0.140 | 0.048 | 0.172 | 0.076 | 0.542 | 0.040 | 0.280 | 0.123 | 0.134 | 0.000 | 0.197 | 0.080 |
| 16.186 | 1.467 | 0.105 | 0.140 | 0.048 | 0.197 | 0.077 | 0.726 | 0.051 | 0.318 | 0.105 | 0.166 | 0.011 | 0.223 | 0.090 |
| 18.209 | 1.641 | 0.124 | 0.165 | 0.067 | 0.222 | 0.086 | 0.892 | 0.062 | 0.350 | 0.111 | 0.185 | 0.022 | 0.261 | 0.105 |
| 20.233 | 1.757 | 0.105 | 0.172 | 0.057 | 0.235 | 0.077 | 1.045 | 0.062 | 0.388 | 0.111 | 0.210 | 0.038 | 0.306 | 0.133 |
| 22.256 | 1.828 | 0.085 | 0.191 | 0.057 | 0.254 | 0.077 | 1.185 | 0.067 | 0.433 | 0.122 | 0.236 | 0.048 | 0.350 | 0.144 |
| 24.279 | 1.879 | 0.076 | 0.203 | 0.058 | 0.273 | 0.077 | 1.293 | 0.081 | 0.477 | 0.133 | 0.287 | 0.066 | 0.401 | 0.167 |
| 26.303 | 1.931 | 0.076 | 0.222 | 0.058 | 0.299 | 0.079 | 1.415 | 0.102 | 0.528 | 0.139 | 0.319 | 0.077 | 0.439 | 0.200 |
| 28.326 | 1.963 | 0.066 | 0.235 | 0.048 | 0.318 | 0.079 | 1.523 | 0.099 | 0.592 | 0.150 | 0.344 | 0.083 | 0.484 | 0.213 |
| 30.349 | 1.982 | 0.066 | 0.267 | 0.051 | 0.349 | 0.090 | 1.618 | 0.118 | 0.649 | 0.166 | 0.383 | 0.100 | 0.522 | 0.232 |
| 32.373 | 2.008 | 0.076 | 0.280 | 0.040 | 0.369 | 0.090 | 1.708 | 0.129 | 0.713 | 0.177 | 0.421 | 0.119 | 0.567 | 0.242 |
| 34.396 | 2.021 | 0.076 | 0.299 | 0.040 | 0.394 | 0.112 | 1.790 | 0.137 | 0.764 | 0.183 | 0.453 | 0.135 | 0.611 | 0.253 |
| 36.419 | 2.053 | 0.066 | 0.337 | 0.044 | 0.419 | 0.101 | 1.867 | 0.150 | 0.821 | 0.199 | 0.504 | 0.172 | 0.643 | 0.261 |

TABLE 3
Amount of ethanol produced by yeast expressing AGT1.

| | Strain/vector | | | | | |
|---|---|---|---|---|---|---|
| | 1848/ | 802/ | 5464/ | 1848/ | 802/ | 5464/ |
| | EV¹ | EV¹ | EV¹ | AGT1² | AGT1² | AGT1² |
| Average ethanol | 0.11 | 0.19 | 0.14 | 0.21 | 0.40 | 0.23 |
| produced | | | | | | |
| (% of volume) | | | | | | |
| Std dev | 0.00 | 0.00 | 0.05 | 0.04 | 0.01 | 0.02 |

¹Average and std dev are the result of two replicates.
²Average and std dev are the result of three replicates.

### Example 4

### Expression of Three Alleles of AGT1 in a ΔAGT1 Strain

In order to separate the effects of the endogenous AGT1 from the transgene, AGT1 alleles were expressed in a strain lacking AGT1. The AGT1 deletion strain from the diploid ORF deletion library (GSA-7) was used. Expression plasmids consisted of three alleles of AGT1 (AGT1Han, AGT11334, and AGT1802) cloned between the promoter and terminator of the triose phosphate isomerase gene (TPI). Additionally the entire gene from 1334, including promoter and terminator (natAGT11334), was cloned. Each AGT1 expression cassette (promoter-CDS-terminator) was cloned into two plasmids, both of which have the ura3 gene as a selectable marker and were derived from the plasmids p416 MET25 and p426 MET25 by replacement of the expression cassette. p416 MET25 has a CEN/ARS yeast origin of replication, which maintains a single copy of the plasmid per cell. p426 MET25 has a 2μ origin of replication, for multiple copy number of the plasmid per cell. EV corresponds to empty vector control. Average and std dev are the result of three replicates. A positive control (strain 1334) and a negative control (Ethanol Red) were not done in replicates.

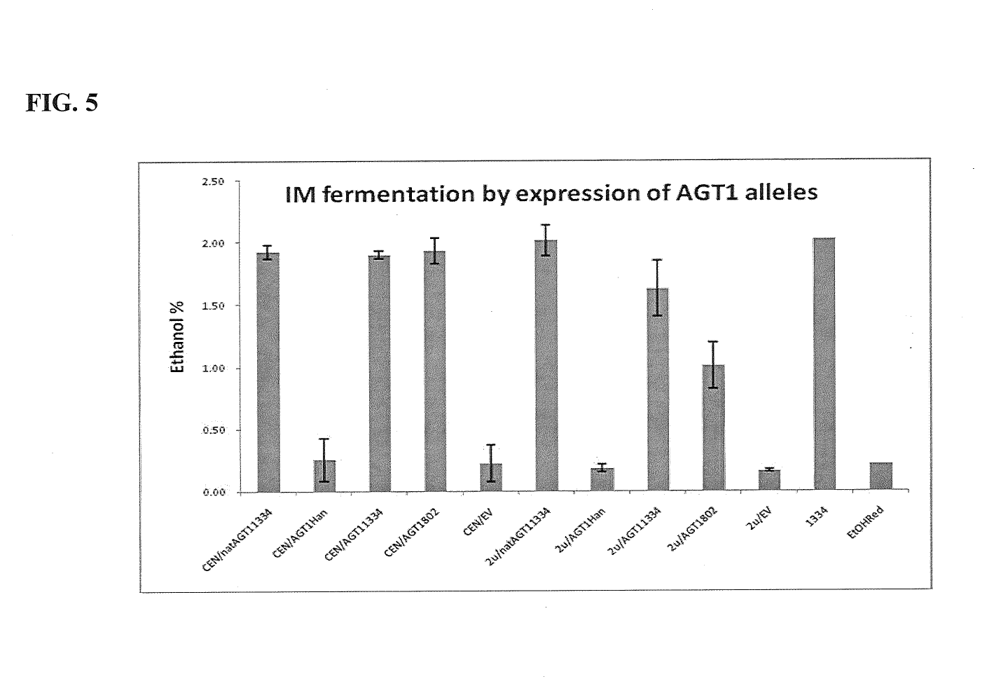

It was found that natAGT11334, AGT11334, and AGT1802, but not AGT1Han, were able to confer IM-fermentation phenotype in the AGT1-deficient strain (FIG. 5 and Table 4) but there were noticeable differences in the total amount of ethanol produced as well as in the rate of fermentation among the strains depending on the expression cassette used. Because plasmids carrying the AGT1Han allele did not produce significant amounts of ethanol it is not discussed in the paragraph below.

TABLE 4
Amount of ethanol produced by yeast
expressing different alleles of AGT1.

| Vector | Ethanol (% vol) | Std dev |
|---|---|---|
| CEN/natAGT1₁₃₃₄ | 1.92 | 0.05 |
| CEN/AGT1_{Han} | 0.26 | 0.17 |
| CEN/AGT1₁₃₃₄ | 1.90 | 0.03 |
| CEN/AGT1₈₀₂ | 1.93 | 0.10 |
| CEN/EV | 0.23 | 0.15 |
| 2μ/natAGT1₁₃₃₄ | 2.02 | 0.12 |
| 2μ/AGT1_{Han} | 0.19 | 0.03 |
| 2μ/AGT11₃₃₄ | 1.63 | 0.22 |
| 2μ/AGT1₈₀₂ | 1.01 | 0.18 |
| 2μ/EV | 0.17 | 0.01 |

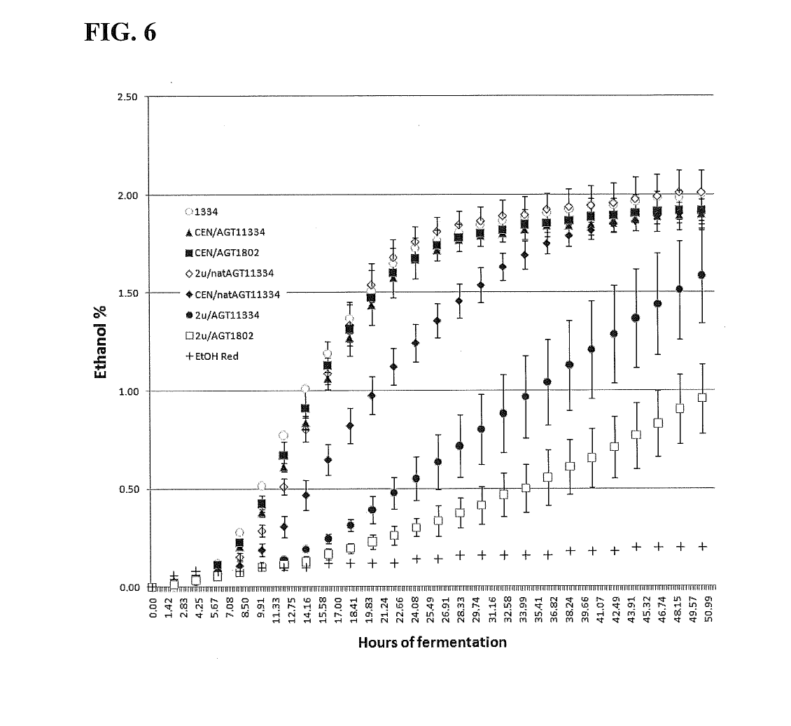

Yeast carrying multiple copy plasmids overexpressing AGT1 (2μ/AGT1₁₃₃₄ and 2μ/AGT1₈₀₂) produced significantly less ethanol than the top-producers and fermentation of IM in those strains proceeded much slower (FIG. 6). At the other extreme, the best performers in terms of final ethanol produced and its fermentation rate were the strains overexpressing AGT1 from a single copy plasmid (CEN/AGT1₁₃₃₄ and CEN/AGT1₈₀₂) and yeast carrying multiple copies of natAGT1₁₃₃₄ (2μ/natAGT1₁₃₃₄). Yeast carrying a single copy of natAGT1₁₃₃₄ (CEN/natAGT1₁₃₃₄) fermented about the same amount as the best but did so at a slower rate.

The data shows that levels of AGT1 can be increased, to a certain point, in order to obtain faster IM fermentation through either gene copy number or promoter strength. However, above a certain threshold, additional AGT1 is detrimental for IM fermentation, perhaps reflecting a negative metabolic effect resulting from too much AGT1.

The amino acid alignment of alleles of AGT1 in Example 2 shows that AGT1_{Han} carries an insertion of an amino acid in addition to three non-conserved substitutions with respect to AGT1₈₀₂ and AGT1₁₃₃₄. The amino acid alterations are due to a pair of nucleotide insertions in the AGT1 gene as shown below, generating a frame shift and extra amino acids. The amino acids in the altered area are highly conserved and are likely the reason for the loss of function of AGT1_{Han}.

| (SEQ ID NO: 12) | | |
|---|---|---|
| | | 1172 1214 |
| AGT1 802 | (1172) | CATATTTTTTTGAAAG--AGCAGGTA-TGGCCACCGACAAGGC |
| (SEQ ID NO: 13) | | |
| AGT1 Han | (1172) | CATATTTTTTTGAAAAGAAGCAGGTAATGGCCACCGACAAGGC |

### Example 5

### Fermentation of Panose

Two strains of Saccharomyces cerevisiae were tested for panose fermentation, Ethanol Red and 1334. 1 ml of overnight yeast culture (yeast peptone base, 4% isomaltulose) was spun down and the pellet resuspended in 1 ml of 4% panose in a 1.5 ml eppendorf tube. Samples were incubated overnight, ˜16 hours, after which they were centrifuged to pellet the cells and the supernatants were taken for carbohydrate analyses. Carbohydrate separation and detection was done with a Dionex IC3000 system with a Dionex AS autosampler, a Dionex DC detection compartment (pulsed amperometric detection (PAD) using a disposable Dionex carbohydrate certified gold surface electrode), and a Dionex SP pump system. For high resolution separation, one Carbopac PA200 3×50 mm Guard Column followed by one Carbopac PA200 3×250 mm analytical column were used for analysis. The electrode potentials were set to the carbohydrates standard quad with AgCl reference electrode as specified by Dionex Corporation. The eluent system utilized an isocratic mobile phase consisting of 100 mM NaOH and a gradient from 0 to 900 mM to 0 NaOAc with a 30 min run time. Peak identification was based on standard retention time of panose (Sigma). Peak analysis utilized Chromeleon version 7.0 software (Dionex Corp., Sunnyvale, Calif.).

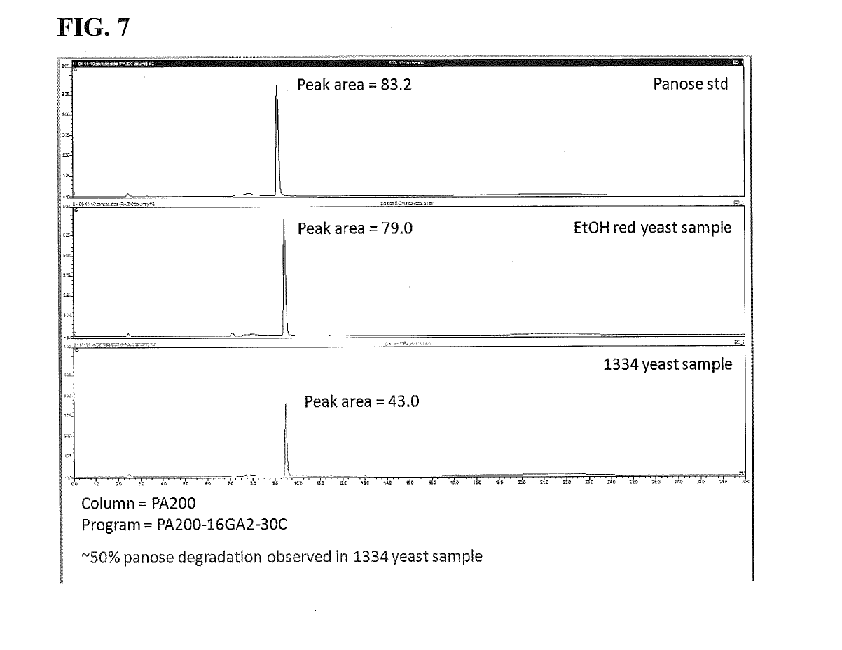

The results show that strain 1334 is capable of fermenting panose (FIG. 7). Under these conditions, 1334 degraded about 50% of the panose in the sample.

The foregoing is illustrative of the present invention, and is not to be construed as limiting thereof. The invention is defined by the following claims, with equivalents of the claims to be included therein.

1. A method of fermenting an oligosaccharide to produce ethanol, comprising contacting the oligosaccharide with a recombinant yeast cell comprising a heterologous polynucleotide encoding a yeast AGT1 polypeptide;
wherein the yeast AGT1 polypeptide comprises an amino acid sequence that is at least 98% identical to the amino acid sequence of SEQ ID NO:1 or an N-terminal fragment thereof of at least about 590 amino acids.

2. The method of claim 1, wherein the polynucleotide is in an expression vector.

3. The method of claim 2, wherein the expression vector maintains a single copy per cell.

4. (canceled)

5. The method of claim 2, wherein the expression vector maintains multiple copies per cell.

6. (canceled)

7. The method of claim 1, wherein the polynucleotide is integrated into the genome of the recombinant yeast cell.

8. The method of claim 1, wherein the AGT1 polypeptide comprises the amino acid sequence of SEQ ID NO:1 or SEQ ID NO:3.

9. (canceled)

10. The method of claim 1, wherein the recombinant yeast cell does not comprise a functional endogenous AGT1 gene.

11. The method of claim 1, wherein the recombinant yeast cell is from a strain selected from the group consisting of Saccharomyces, Schizosaccharomyces, Kluyveromyces, Trichosporon, Schwanniomyces, Pichia, Hansenula, Arxula, Candida, Kloeckera, and Yarrowia.

12. (canceled)

13. The method of claim 1, wherein the oligosaccharide is a disaccharide or trisaccharide.

14. The method of claim 1, wherein the oligosaccharide is selected from the group consisting of isomaltulose, trenalulose, maltose, panose, and maltotriose.

15. (canceled)

16. (canceled)

17. The method of claim 1, wherein the oligosaccharide is obtained from plant material.

18. The method of claim 17, wherein the plant material is from maize, sugar beet, sorghum, or sugarcane.

19. The method of claim 1, wherein the amount of ethanol produced during fermentation reaches half maximum within 15 hours of contacting the oligosaccharide.

20. (canceled)

21. A method of modifying a yeast cell to decrease lag time for ethanol production and/or increase the amount of ethanol production during fermentation of an oligosaccharide, comprising inserting into the yeast cell a polynucleotide encoding a yeast AGT1 polypeptide;
wherein the yeast AGT1 polypeptide comprises an amino acid sequence that is at least 98% identical to the amino acid sequence of SEQ ID NO:1 or an N-terminal fragment thereof of at least about 590 amino acids.

22-34. (canceled)

35. A recombinant yeast cell for production of ethanol from an oligosaccharide, the recombinant yeast cell comprising a heterologous polynucleotide encoding a yeast AGT1 polypeptide;
wherein the yeast AGT1 polypeptide comprises an amino acid sequence that is at least 98% identical to the amino acid sequence of SEQ ID NO:1 or an N-terminal fragment thereof of at least about 590 amino acids.

36. The recombinant yeast cell of claim 35, wherein the polynucleotide is in an expression vector.

37. The recombinant yeast cell of claim 36, wherein the expression vector maintains a single copy per cell.

38. (canceled)

39. The recombinant yeast cell of claim 36, wherein the expression vector maintains multiple copies per cell.

40. (canceled)

41. The recombinant yeast cell of claim 35, wherein the polynucleotide is integrated into the genome of the recombinant yeast cell.

42. The recombinant yeast cell of claim 35, wherein the AGT1 polypeptide comprises the amino acid sequence of SEQ ID NO:1 or SEQ ID NO:3.

43. (canceled)

44. The recombinant yeast cell of claim 35, wherein the recombinant yeast does not comprise a functional endogenous AGT1 gene.

45. The recombinant yeast cell of claim 35, wherein the recombinant yeast cell is from a strain selected from the group consisting of Saccharomyces, Schizosaccharomyces, Kluyveromyces, Trichosporon, Schwanniomyces, Pichia, Hansenula, Arxula, Candida, Kloeckera, and Yarrowia.

46. (canceled)
